(19) United States
(12) Patent Application Publication
(10) Pub. No.: US 2026/0174524 A1
(43) Pub. Date: Jun. 25, 2026

(54) TEMPLATE FOR POSITIONING HOLES IN DAMS USED FOR ABSOLUTE DENTAL ISOLATION

(71) Applicant: Victor Daniel Guerrero Munive, Lima (PE)

(72) Inventor: Victor Daniel Guerrero Munive, Lima (PE)

(73) Assignee: Victor Daniel Guerrero Munive, Lima (PE)

(21) Appl. No.: 19/125,778

(22) PCT Filed: Sep. 1, 2023
(86) PCT No.: PCT/PE2023/050012

§ 371 Date: Apr. 30, 2025

(30) Foreign Application Priority Data
Nov. 16, 2022 (PE) 002665-2022/DIN

(51) Int. Cl. A61C 1/08; A61B 17/17; A61C 7/14; A61C 13/00; A61C 13/12

(52) U.S. Cl. CPC A61C 1/084; A61B 17/176; A61C 7/146; A61C 13/0004; A61C 13/12

(57) ABSTRACT

A template for positioning perforations to be made in dams, characterized by standardized markings for well-aligned dental arches and a curved millimeter ruler to customize the position for perforations in irregular dental arches, as well as coordinate lines and midlines, which allow tooth segmentation to be performed to position the perforation. This template allows for varying positions and distances when making perforations in latex and silicone (synthetic) dams, facilitating its use in patients with irregularly aligned dental arches. It can also be used on dams of different thicknesses (thin, medium, and thick), especially since it facilitates varying the distances and positions of the perforations.

### TECHNICAL FIELD

The following invention is referred to a template for positioning perforations to be made in latex and synthetic dams in the healthcare field, measuring “6×6” and “5×5” inches, specifically related to oral health. This template serves as a more precise and individualized guide when applying the ‘absolute Isolation’ technique, a method used to isolate teeth from the patient's mouth and optimize dental procedures. It also serves as a biosecurity method to avoid the aerosol production in the mouth when using dental rotary instruments.

### BACKGROUND OF THE INVENTION

As Tanalp J M (2014) stated, one of the main reasons why dentists and dental students discourage using this technique in daily dental care is the difficulty of correctly placing the dam (latex or synthetic) in the patient's mouth. Currently, there are templates for dam perforations from various companies, but they only consider the standard tooth locations with fixed positions and distances.

Some of the templates available on the market are the following:
a) https://www.dentaltix.com/es/hygenic/plantilla-impresa
   Known as the “Printed Template-Hygenic,” this template is used as a guide when marking the position of the holes in the dental dam, as indicated by the manufacturer. The template itself shows only the standard teeth locations and is for the 6×6-inch dam size.
b) https://www.dentalsegurite.es/producto/plantilla-dique-goma-hu-friedyuniversal/Known
   Known as the “Hu-Friedy Universal Rubber Dam Template,” it belongs to the Hu-Friedy brand. Like the one mentioned above, it only has the standard teeth locations and is for the 6×6-inch dam size. This template also includes a number that identifies each tooth.
c) https://edenta.eu/en/endo-accessories/rubber-dam-template-for-rubber-dam-1-unit
   Known as the “Dental Dan Template,” it is noted that, like the previous templates, it only has the standard tooth placement and is suitable for a 6×6-inch dam.

One drawback that oral health professionals face when using existing templates is that they do not grant variation in tooth positions for more complex cases, such as severely compromised teeth, especially in periodontally affected patients and older adults.

Likewise, they are not easy to use when dealing with different dam thicknesses because the clinical indications for the different thicknesses vary, and the materials on which they are printed are paper and cardboard, which makes them easily damaged.

Therefore, it is necessary to provide a template that solves the problems mentioned above.

### DESCRIPTION OF THE INVENTION

As a solution to the problems mentioned above, the present invention was developed. This is a 15 cm×15 cm template. Unlike commercially available templates that only have fixed positions and distances for the teeth and are designed for a dam thickness, this template allows for varying positions and distances when drilling holes in latex and silicone (synthetic) dams, facilitating its use in patients with irregularly aligned dental arches, diastemas (small spaces between teeth), edentulous spaces, and subgingival caries.

It can also be used on rubber dams of different thicknesses (thin, medium, and thick), especially since it makes it easier to vary the distances and positions of the perforations.

The present invention allows for varying positions and distances when creating the perforations due to its three parts:
a) Coordinate lines (1) and midline (2), according to the invention, the template comprises four coordinate lines (1) distributed in the corners (two horizontal and two vertical). These lines are distributed in the upper and lower corners, with a horizontal and a vertical line, making it easier to vary the position of the template on the dams to facilitate the segmentation of the right and left, upper and lower teeth of the patient's jaws.
   A midline (2) divides the dam in half for anterior teeth (upper and lower). This line coincides with the dental midline, ensuring that the dam is centered within it.
b) Positions with fixed distances that can be marked on the dams (3), this is preferably used on well-aligned dental arches with a medium-thickness latex dam, applied to simple clinical situations where access to the cervical area of the teeth is not required. It is similar to the templates currently available on the market, but does not have an exact match in distances, as each brand sets its own distance for the markings, and there is currently no standard measurement for these distances on the market.
c) A curved millimeter ruler (4), allows the operator (oral health professional) to adjust and customize the position of the perforations in the dam. This ruler features a distance between each marking for anterior teeth (canine to canine) of 5 millimeters (which can also be represented within a range of 3 to 6 mm) and for posterior teeth of 7 millimeters (which can also be represented within a range of 5 to 8 mm). It can be used in more complex clinical situations, such as in patients with irregularly aligned dental arches, diastemas (small spaces between teeth), edentulous spaces, and subgingival caries. In these cases, precision in the position of the perforations made in the dams for absolute isolation is of utmost importance, since this is how moisture control and gingival tissue retraction are achieved. The center of the millimeter ruler can also be empty, closed, or have a removable tab to facilitate its use by the operator.

With the variations made, the template of the present invention surpasses the state of the art since with a single template, the perforations can be located with greater precision in the dams of different thicknesses that are commercially available, and additionally it facilitates work on irregular dental arches.

### BRIEF DESCRIPTION OF THE FIGURES

FIG. 1: Shows a complete view of the proposed template, showing the arch with the standard positions and the arch with the millimeter ruler that facilitates changing positions, the coordinate lines, and the lines that will divide the rubber dam according to the work areas.

FIG. 2: Shows a view of the upper half of the proposed template, which represents the arch with the standard tooth positions in the dental arches.

FIG. 3: Shows a view of the lower half of the proposed template, which represents the arch with the curved millimeter ruler, the major and minor lines, spaced according to the positions of the upper half of the template. It also includes an empty space in the center of the millimeter ruler.

### PREFERRED EMBODIMENT OF THE INVENTION

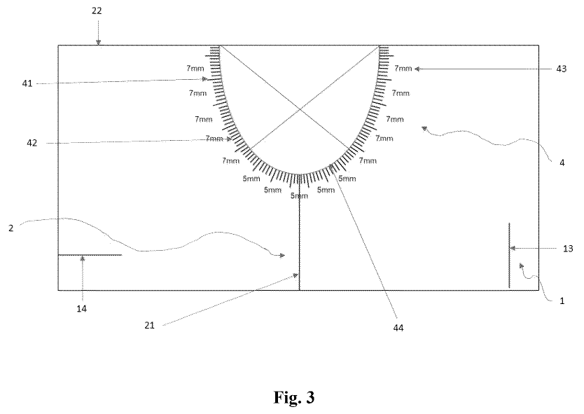

As can be seen in FIG. 3 attached to this document, the template for positioning the perforations in the dams comprises coordinate lines (1), a midline (2), a standardized mark for well-aligned dental arches (3), and a curved millimeter ruler (4). It can be used from left to right and from top to bottom, depending on the needs of the clinical case.

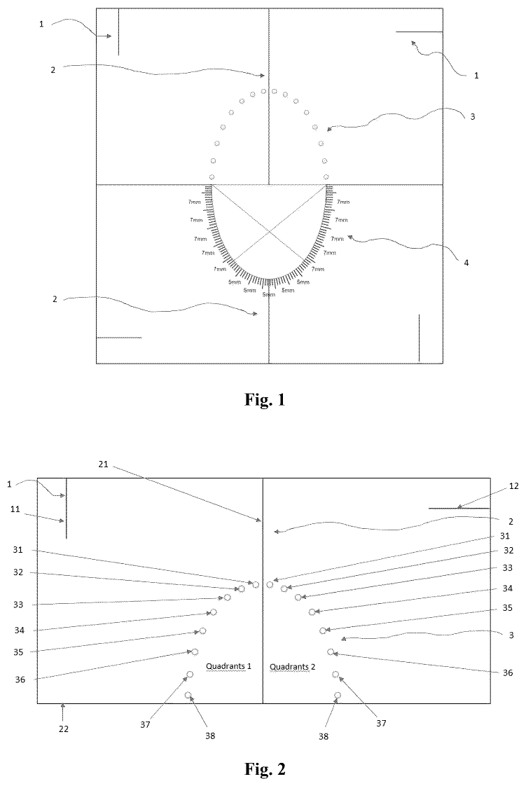

The coordinate lines (1) are located in the upper and lower halves of the template (FIG. 2 and FIG. 3), including the upper vertical line (11), lower vertical line (13), upper horizontal line (12), and lower horizontal line (14). The names are for reference only because the template can also be used inverted. The midline (2) is represented by the vertical midline (21), which divides the dam from left to right and crosses the entire template, and the horizontal midline (22), which divides the dam into two parts: an upper half and a lower half.

The standardized mark for well-aligned dental arches (3), as its name suggests, represents a dental arch for dams of medium thickness. The arch is divided into two quadrants separated by the vertical midline (21), with each hole representing a tooth. The template can be used directly or rotated, reversing its position. The teeth are identified in pairs. The mentioned teeth are represented in FIG. 2 and are right/left central incisor-upper/lower (31), right/left lateral incisor-upper/lower (32), right/left canine-upper/lower (33), right/left first premolar-upper/lower (34), right/left second premolar-upper/lower (35), right/left first molar-upper/lower (36), right/left second molar-upper/lower (37) and right/left third molar-upper/lower (38).

The curved millimeter ruler (4) facilitates position changes in irregular dental arches for dams of any thickness (FIG. 3). It consists of major lines (41) that mark the exact position of the teeth represented in the upper half of the template, minor lines (42) that mark the distance in millimeters between the major lines, reference numbers (43) that are used to identify customized positions, which may or may not be printed on the template, and the dental arch (44), which represents the shape of the dental arch, on which the marks for the dam perforations will be positioned. It is important to note that within the dental arch, there may be an empty space, a closure, or a removable tab, represented by an “X.” The template may be printed on transparent plastic mica, cardboard, or aluminum. It is recommended that for ease of use, handling, and disinfection by the operator, it be preferably printed on transparent plastic laminate.

1. A template for positioning perforations in dams used for the absolute dental isolation technique, comprising markings for well-aligned dental arches (3), characterized in that it also comprises a curved millimeter ruler (4) that customizes the position for perforations in irregular dental arches, and where said template is also made up of: major lines (41) that mark the exact position of the teeth represented in the upper half of the template, minor lines (42) that mark the distance in millimeters between the major lines.

2. The template for positioning perforations in dams used for the absolute dental isolation technique, comprising marks for well-aligned dental arches (3) and a curved millimeter ruler (4) according to claim 1, characterized in that it also comprises coordinate lines (1) located in the upper and lower halves of the template, having the upper vertical line (11), lower vertical line (13), upper horizontal line (12) and lower horizontal line (14), the names are referential because the template can also be used inverted, allowing the position of the template to vary on the dams located in the upper and lower halves of the template, and midlines (2) to align the template with the dental midline, represented by the vertical midline (21) that divides the dam from left to right and crosses the entire template and the horizontal midline (22) that divides the dam into two, an upper half and a lower half.

3. The template for positioning the perforations in the dams used for the absolute dental isolation technique according to claim 1, wherein the millimeter ruler comprises differentiated measurements for the anterior teeth, in a range of 3 mm to 6 mm and for the posterior teeth in a range of 5 mm to 8 mm.

4. The template for positioning perforations in dams, used for the absolute dental isolation technique, comprising markings for well-aligned dental arches (3) and a curved millimeter ruler (4), according to claim 1, characterized in that it also includes a closed empty space or a type of removable tab in the center of the millimeter ruler.

5. Template for positioning perforations in dams, used for the absolute dental isolation technique, comprising markings for well-aligned dental arches (3) and a curved millimeter ruler (4), according to claim 1, characterized in that said template is made of plastic, paper, cardboard, and metal.
